(19) United States
(12) Patent Application Publication
(10) Pub. No.: US 2005/0233465 A1
(43) Pub. Date: Oct. 20, 2005

(54) Compositions of matter useful as pH indicators and related methods

(75) Inventor: Scott E. Miller, Somerville, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: BioProcessors Corp., Woburn, MA (US)

(21) Appl. No.: 11/107,348

(22) Filed: Apr. 14, 2005

Related U.S. Application Data
(60) Provisional application No. 60/561,946, filed on Apr. 14, 2004.

(51) Int. Cl. C07D 221/22; G01N 21/75

(52) U.S. Cl. 436/164; 546/37; 546/38

(57) ABSTRACT

The present invention provides chemical compounds (compositions of matter) and methods useful for determining pH and/or pH change in an environment. Specific compounds of the invention include at least five fused molecular rings.

### RELATED APPLICATIONS

This application claims the benefit of priority under 35 U.S.C. §119(e) to commonly-owned, co-pending U.S. provisional patent application Ser. No. 60/561,946 filed Apr. 14, 2004, entitled “COMPOSITIONS OF MATTER USEFUL AS pH INDICATORS AND RELATED METHODS,” by Scott E. Miller.

### FIELD OF INVENTION

The present invention relates generally to molecules that absorb and/or emit electromagnetic radiation in wavelength ranges making them useful as indicators, and more particularly to molecules useful as pH dyes/indicators and related methods of use.

### BACKGROUND OF INVENTION

pH indicator molecules generally are molecules whose physical properties change depending upon the pH of an environment to which they are exposed, so that the pH of the environment can be determined based upon this change. For example, the molecules may change color depending upon pH, change intensity of absorption or emission of light depending upon pH, or both. As an example, some pH indicator molecules are fluorescent molecules which absorb light at a particular wavelength and emit light at a second, longer wavelength. Their pH indicating function typically involves protonation and deprotonation. This means that these fluorescent pH indicators include a hydrogen atom (also sometimes referred to as a “proton,” symbolized by H⁺) which forms part of the molecule (is bound to the molecule) in one pH range, but within another pH range the proton is dissociated from the molecule. When the proton is disassociated from the molecule, the molecule takes on a negative charge, which is balanced by a positively-charged ion (e.g., Na⁺) in solution with the indicator. This arrangement is illustrated by Eq. 1.
R—H⇄R⁻+H⁺ Equation 1

Eq. 1 represents an equilibrium where, under certain conditions, the equilibrium exists predominantly towards the left side of the equation, with R—H predominantly present, and under other conditions the equilibrium exists predominantly toward the right, with R⁻ predominantly present, from which H⁺ has become dissociated. This equilibrium is described mathematically by Eq. 2
Kₐ=[H⁺][R⁻]/[R—H] Equation 2

[H⁺], [R⁻], and [R—H] are the equilibrium concentrations in moles/liter of H+, R— and R—H, respectively, and Kₐ is the acid dissociation constant of the molecule in moles/liter. At a given concentration of H⁺ (pH), the relative amounts of R⁻ and R—H existing in equilibrated solution must satisfy Equation 2.

Where RH is a pH indicator molecule, the equilibrium can be shifted toward the left or toward the right by the pH of the solution in which RH is present. At low pH, with an abundance of H⁺ ions present, the equilibrium will be shifted toward the left. In a high pH environment, with less H⁺ present, the equilibrium will be shifted toward the right. Where R represents a fluorescent molecule, it generally will exhibit fluorescence at a different wavelength (will be visible as a very different color) based upon whether it is in the R—H form or in the R⁻ form. For most molecules represented by R, this change will occur generally quite abruptly within a very narrow pH range, allowing R to serve as a very simple and reliable pH indicator. When placed in solution, it will exhibit one very distinct color (a color associated with its R—H form), and another very distinct color associated with its R⁻. Other pH indicator molecules, depending upon the pH of the environment to which they are exposed and therefore their relative position in the equilibrium of Eq. 1, will not change in wavelength emission, but will change in emission intensity. Other molecules will change in both characteristics.

pH indicators can be used in a liquid such as an aqueous solution, added dropwise to a solution to monitor pH, or can be impregnated or otherwise associated with a piece of paper, forming the commonly-known “pH strips” which are dipped in liquid to determine pH. The most common pH strip is a red/blue pH strip, which is a piece of paper impregnated with a molecule that exhibits a red fluorescence in an acid (where the molecule is predominantly in its R—H form), and blue in a base (where the molecule is predominantly in its R⁻ form).

While a variety of molecules suitable as pH indicators are known, improved and varied indicators would be useful.

### SUMMARY OF INVENTION

The present invention involves a new set of pH indicator molecules. The molecules can be used in any of a wide variety of situations in which pH is desirably indicated, and they find particular use in certain environments in which the effectiveness of some previously-known indicator molecules is hindered by inherent absorption of the environment within the wavelength range at which the indicator is active.

That is, in one aspect the invention involves the recognition that many previously-known indicator molecules are hindered in that the spectrum in which they are active is largely coincident with absorption of biological media. The invention provides, as a solution, indicator molecules that are particularly useful in environments having significant inherent absorption of electromagnetic radiation (e.g., light) within a wavelength range at which the pH indicator molecule absorbs or emits electromagnetic radiation differently based upon the pH of the environment. The inherent absorption of radiation by the environment thereby can obscure the emission and/or absorption properties of the pH indicator molecule.

Biological media, i.e., environments that contain biological species, environments that mimic biological species' environments, and/or fluids that supply nutrients or the like to biological environments or remove products from biological environments, can be particularly useful environments within which pH indicator molecules of the invention can function, because these environments generally absorb significant light in regions that typical previously-known pH indicators operate. In one aspect, the invention provides a series of molecules, or compositions of matter. In one embodiment, the invention provides a compound comprising at least five fused molecular rings, the compound having the ability to be protonated or deprotonated as a result in a change in pH of a medium to which the compound is exposed. In one set of embodiments, the compound comprises at least five fused organic molecular rings having significant delocalization of pi-electron structure (for example, aromatic molecular structure), such that the compound absorbs and/or emit electromagnetic radiation significantly at wavelengths greater than 400 nm or 450 nanometers. The compound absorbs at greater than 400 nm or 450 nanometers with a molar absorptivity at at least 5000/mole.cm, and/or emit at greater than 400 nm or 450 nanometers with a quantum yield of at least 5%. These compounds are generally hydrocarbon molecules but can include heteroatoms in place of carbons (of a purely hydrocarbon structure) such as oxygen (O) and nitrogen (N).

In another aspect, the invention provides a series of methods. One method of the invention involves providing a chemical compound comprising at least five fused molecular rings, or other compound described herein, exposing the chemical compound to an environment at a pH, and determining the pH of the environment by determining an interaction of the chemical compound with electromagnetic radiation.

The subject matter of this application may involve, in some cases, interrelated products, alternative solutions to a particular problem, and/or a plurality of different uses of a single system or article.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

FIG. 1 shows the absorption spectrum of cell culture media (solid line) and the absorption spectrum of 8-hydroxypyrene-1,3,6-trisulfonic acid (HPTS), a known dye, combined with the cell culture media (dashed line).

FIG. 2 shows representative absorption spectra for a series of increasingly larger prophetic aromatic molecules demonstrating the principle of the present invention.

FIG. 3 shows a schematic representation of the synthesis of one embodiment of the present invention.

### DETAILED DESCRIPTION

Each of the following applications is incorporated herein by reference: U.S. Provisional Patent Application Ser. No. 60/282,741, filed Apr. 10, 2001, entitled “Microfermentor Device and Cell Based Screening Method,” by Zarur, et al.; U.S. patent application Ser. No. 10/119,917, filed Apr. 10, 2002, entitled “Microfermentor Device and Cell Based Screening Method,” by Zarur, et al.; International Patent Application No. PCT/US02/11422, filed Apr. 10, 2002, entitled “Microfermentor Device and Cell Based Screening Method,” by Zarur, et al., published as WO 02/083852 on Oct. 24, 2002; U.S. Provisional Patent Application Ser. No. 60/386,323, filed Jun. 5, 2002, entitled “Materials and Reactors having Humidity and Gas Control,” by Rodgers, et al.; U.S. Provisional Patent Application Ser. No. 60/386,322, filed Jun. 5, 2002, entitled “Reactor Having Light-Interacting Component,” by Miller, et al.; U.S. patent application Ser. No. 10/223,562, filed Aug. 19, 2002, entitled “Fluidic Device and Cell-Based Screening Method,” by Schreyer, et al.; U.S. Provisional Patent Application Ser. No. 60/409,273, filed Sep. 24, 2002, entitled “Protein Production and Screening Methods,” by Zarur, et al.; U.S. patent application Ser. No. 10/457,048, filed Jun. 5, 2003, entitled “Reactor Systems Responsive to Internal Conditions,” by Miller, et al.; U.S. patent application Ser. No. 10/456,934, filed Jun. 5, 2003, entitled “Systems and Methods for Control of Reactor Environments,” by Miller, et al.; U.S. patent application Ser. No. 10/456,133, filed Jun. 5, 2003, entitled “Microreactor Systems and Methods,” by Rodgers, et al.; U.S. patent application Ser. No. 10/457,049, filed Jun. 5, 2003, entitled “Materials and Reactor Systems having Humidity and Gas Control,” by Rodgers, et al,. published as 2004/0058437 on Mar. 25, 2004; International Patent Application No. PCT/US03/17816, filed Jun. 5, 2003, entitled “Materials and Reactor Systems having Humidity and Gas Control,” by Rodgers, et al., published as WO 03/103813 on Dec. 18, 2003; U.S. patent application Ser. No. 10/457,015, filed Jun. 5, 2003, entitled “Reactor Systems Having a Light-Interacting Component,” by Miller, et al., published as 2004/0058407 on Mar. 25, 2004; International Patent Application No. PCT/US03/18240, filed Jun. 5, 2003, entitled “Reactor Systems Having a Light-Interacting Component,” by Miller, et al., published as WO 03/104384 on Dec. 18, 2003; U.S. patent application Ser. No. 10/457,017, filed Jun. 5, 2003, entitled “System and Method for Process Automation,” by Rodgers, et al.; U.S. patent application Ser. No. 10/456,929, filed Jun. 5, 2003, entitled “Apparatus and Method for Manipulating Substrates,” by Zarur, et al.; International Patent Application No. PCT/US03/25956, filed Aug. 19, 2003, entitled “Determination and/or Control of Reactor Environmental Conditions,” by Miller, et al., published as WO 2004/016727 on Feb. 26, 2004; U.S. patent application Ser. No. 10/664,046, filed Sep. 16, 2003, entitled “Determination and/or Control of Reactor Environmental Conditions,” by Miller, et al.; International Patent Application No. PCT/US03/25907, filed Aug. 19, 2003, entitled “Systems and Methods for Control of pH and Other Reactor Environmental Conditions,” by Miller, et al., published as WO 2004/016729 on Feb. 26, 2004; U.S. patent application Ser. No. 10/664,068, filed Sep. 16, 2003, entitled “Systems and Methods for Control of pH and Other Reactor Environmental Conditions,” by Miller, et al.; International Patent Application No. PCT/US03/25943, filed Aug. 19, 2003, entitled “Microreactor Architecture and Methods,” by Rodgers, et al.; U.S. patent application filed on Sep. 16, 2003, entitled “Microreactor Architecture and Methods,” by Rodgers, et al.; and International Patent Application Serial No. PCT/US01/07679, published on Sep. 20, 2001 as WO 01/68257, entitled “Microreactors.”

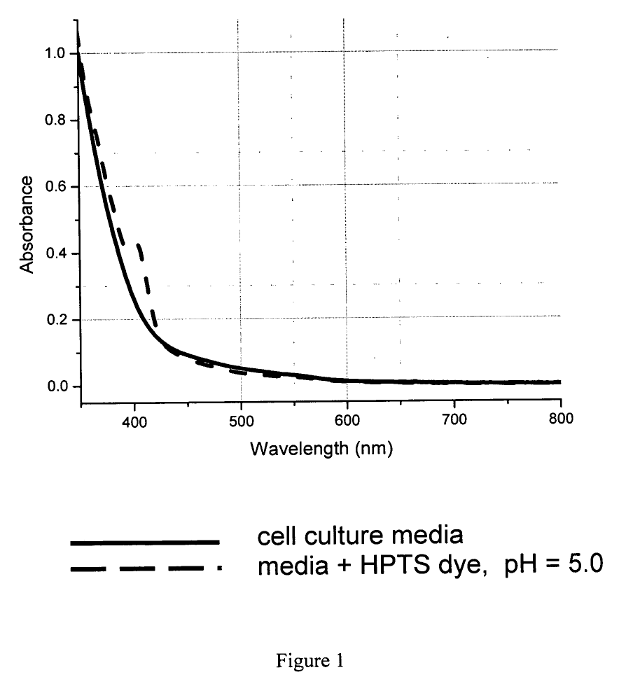

The present invention involves, in one aspect, the recognition that the use of many pH indicator molecules is hindered within certain media (environments) important in the field of chemistry and, especially biology. In particular, the inventors have recognized that many pH indicator molecules are less useful in media which have significant absorbance of electromagnetic radiation at the shorter wavelength end of the visible spectrum, for example, strong absorbance in the 375-475 nm range. Specifically, media for cell culturing (various nutrients needed by cells, in water, sometimes in combination with products produced or expelled by cells themselves into this water) typically exhibit significant absorption of light below 500 nm, with absorption increasing as wavelength is shorter to the point that very significant absorption takes place below 400 nm, as observed in the absorption spectrum in FIG. 1 (solid line). pH indicators known in the art, such as 8-hydroxypyrene-1,3,6-trisulfonic acid or HPTS, predominantly operate within this wavelength range. For example, FIG. 1 shows the absorption spectrum of HPTS combined with media for cell culturing (dashed line), wherein the HPTS absorbance is substantially obscured by the absorbance of the background biological media. Consequently, the indicating properties of HPTS and other pH indicators known in the art can be compromised by the fact that the signal they produce (absorbance/fluorescence) can be masked by absorbance of light of the medium within which they are placed, at wavelengths competing with the indicator wavelengths.

Accordingly, the inventors have recognized the need for pH indicator molecules with indicating activity at wavelengths suitable for use with cell culture media. The present invention provides such molecules, and methods for use of such molecules. While the molecules and methods are useful in connection with cell culture media and other biological environments, the invention is not limited in this way. The molecules and techniques provided herein can be used in essentially any environment in which pH is desirably determined.

In one aspect, the invention provides a chemical compound (composition of matter) comprising at least five fused molecular rings, the compound having the ability to be protonated or deprotonated as a result in a change in pH of a medium to which the compound is exposed. The molecule is preferably selected such that it can serve as a visibly detectable pH indicator, for example an indicator allowing pH assessment with the unaided human eye. In one set of embodiments, the compound comprises at least five fused organic molecular rings having significant delocalization of pi-electron structure (for example, aromatic molecular structure), such that the compound absorbs and/or emit electromagnetic radiation significantly at wavelengths greater than 400 nm or 450 nanometers. The compound absorbs at greater than 400 nm or 450 nanometers with a molar absorptivity at at least 5000/mole.cm, and/or emit at greater than 400 nm or 450 nanometers with a quantum yield of at least 5%. These molecules are generally hydrocarbon molecules but can include heteroatoms in place of carbons (of a purely hydrocarbon structure) such as oxygen (O) and nitrogen (N). It is to be understood that wherever a molecular structure is described herein including “five fused molecular rings, the compound having the ability to be protonated or deprotonated as a result in a change in pH of a medium to which the compound is exposed”, “five (or more) fused rings”, or the like, the structure can be as described more specifically above, for example with significant aromatic structure.

Non-limiting examples of molecules which can be provided, in accordance with the invention, with the ability to be protonated and deprotonated in response to pH of a surrounding medium in a readily-determinable manner, include the following: 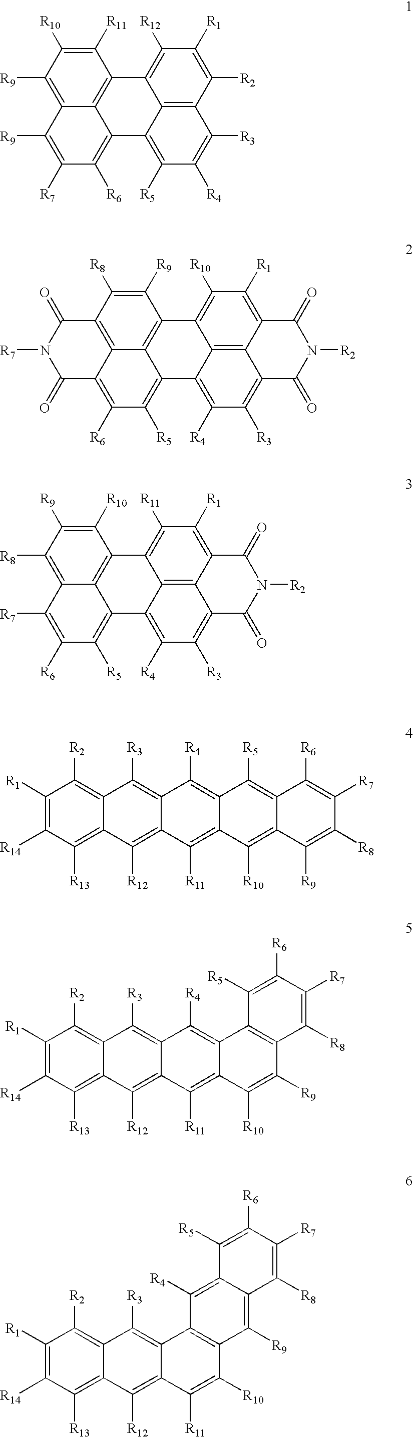 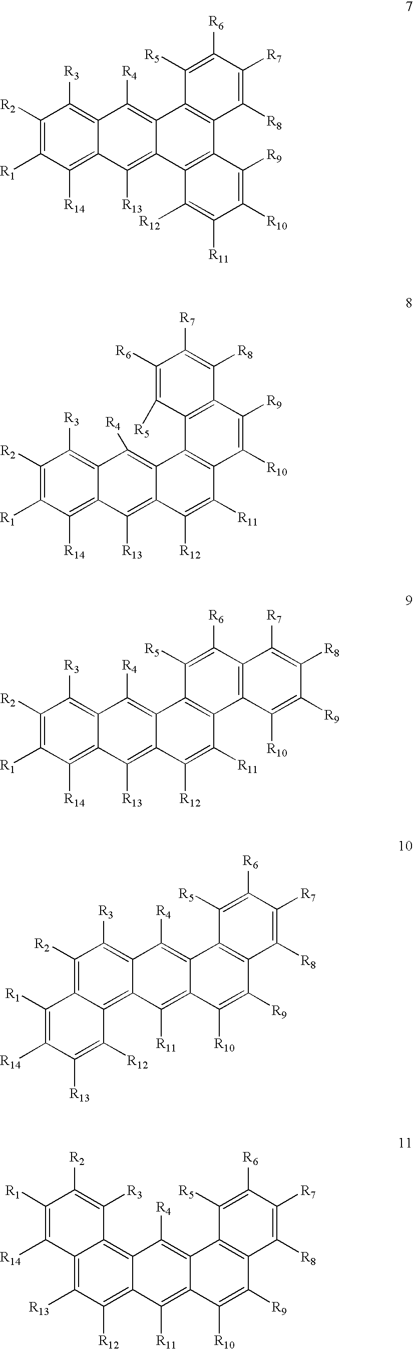 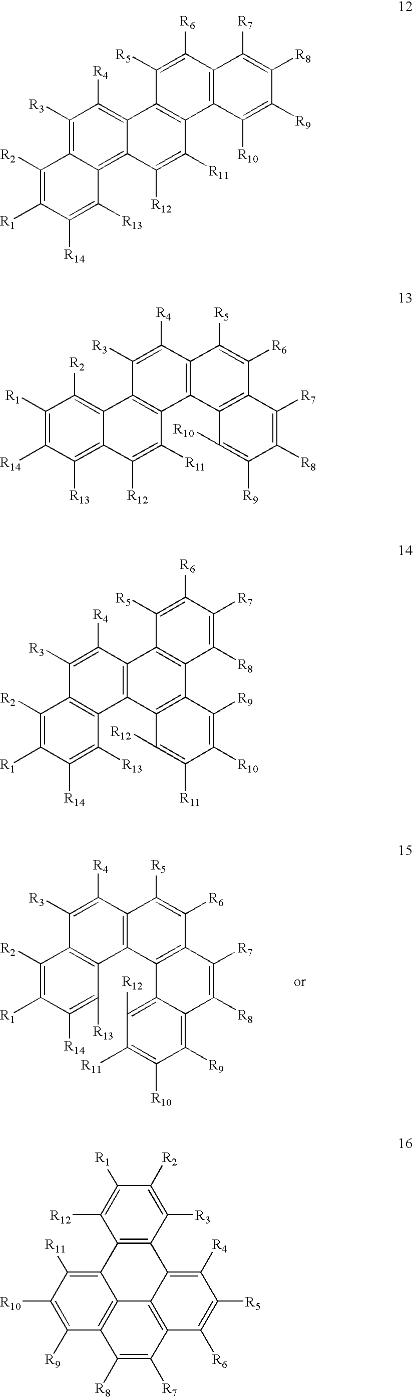
Each R shown above independently can be hydrogen or a functional group or other organic moiety, such as an alkyl group or an aromatic group, an acrylamide, a carboxylic acid, and activated ester of a carboxylic acid, a hydroxyl, an aldehyde, an alkyl halide, a sulfonate, an amine, an anhydride, an aniline, an aryl halide, an azide, an aziridine, a boronate, a carbodiimide, and epoxide, a glycol, an haloacetamide, a halotrazine, a hydrazine, a hydroxylamine, an isothiocyanate, an isocyanate, a thiocarbamate, a ketone, a maleimide, a sulfonyl halide, a thiol group, sulfomethyl, halomethyl, or C₁-C₁₈ or C₁-C₁₈ perfluoroalkyl.

In one embodiment, R is L-S_{c}, wherein L is a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds, and S_{c} can be a metallic or semiconductor nanoparticle, a fullerene, a carbon nanotube, an amino acid, a tyramine, a peptide, a protein, a monosaccharide, a polysaccharide, an ion-complexing moiety, a nucleoside, a nucleotide, an oligonucleotide, a nucleic acid, a hapten, a drug, a lipid, a phospholipid, a lipoprotein, a lipopolysaccharide, a liposome, a lipophilic polymer, a polymeric microparticle, an animal cell, a plant cell, a bacterium, a yeast, a virus, or can comprise one or more additional dye compounds, which may be the same or different. In one embodiment, S_{c} comprises one or more additional compounds which may quench the fluorescence of the compound. In another embodiment, S_{c} comprises one or more additional compounds which may undergo energy transfer to or from compound.

In another embodiment, R is L-Rₓ, wherein L is a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds, and Rₓ can be a succinimidyl ester.

In certain instances, two or more R's together in the structures above may define a cyclic moiety and/or a conjugated group.

In one set of embodiments of the invention, the compound includes a conjugated group. A “conjugated group,” as used herein, refers to an interconnected chain of at least three atoms, each atom participating in delocalized pi-bonding. For example, the chain of three atoms may be a chain of three carbon atoms participating in delocalized pi-bonding, a chain of four or more carbon atoms participating in delocalized pi bonding, a ring of carbon atoms (optionally including nitrogen atoms or the like) participating in delocalized pi bonding, two carbon atoms and a nitrogen atom participating in delocalized pi bonding, etc. In some cases, the conjugated group includes at least one aromatic structure, for example, a benzene ring or a pyridine ring. As used herein, “aromatic” is given its ordinary definition as used in the field of organic chemistry. Other non-limiting examples of aromatic structures include naphthalene rings, anthracene rings, pyridine rings, quinoline rings, thiophene rings, furans, quinolizine rings, coumarins, etc.

Specific examples of molecules of the invention include perylene, perylene diimide, perylene monoimide, and pentacene, coronene (including the mono- and di-imides), terrylenes, quarterrylenes, and derivatives, having the ability to be protonated and deprotonated based upon pH of a surrounding medium in a readily-determinable manner. Those of ordinary skill in the art will clearly recognize the meaning of “can be protonated or deprotonated as a result of change in pH of a surrounding medium in a readily-determinable manner,” and can easily synthesize such molecules without undue experimentation. Protonation and deprotonation can take place via addition and removal of a hydrogen atom from a functional group linked to essentially any portion of the molecule, and examples of such functional groups include hydroxyl groups, amino groups, carbonyl groups, carboxylic acids, and the like. This protonation/deprotonation is readily determinable if, e.g., it causes a change in the electronic structure of the molecule such that emission, absorption, or both is affected significantly, oxidation potential of the molecule is affected, etc. Typically, protonation and deprotonation changes the gap between the highest occupied molecular orbital and the lowest unoccupied molecular orbital (HOMO-LUMO gap) of the molecule, changing absorbance, fluorescence maxima and oxidation potential of various molecule.

In some cases, molecules having larger aromatic cores (for example, having 5, 6, or more fused rings) will have longer absorption and emission maxima, which may make the molecules less susceptible to optical interferences. For example, 8-hydroxypyrene-1,3,6-trisulfonic acid (HPTS) with four fused rings has an absorption maximum of approximately 405 nm. Replacing the pyrene core of HPTS with a perylene core as in compound 1 or a pentacene core as in compound 4 will shift the absorption spectra to longer wavelengths by an estimated 50 nm relative to HPTS, providing greater separation between the absorption spectrum of the indicator and the absorption spectrum of the biological media, e.g. cell culture media. An unsubstituted perylene monoimide (as in compound 3) exhibits absorption and emission maxima at substantially longer wavelengths (λ_{abs, max}=510 nm, 550 nm and λ_{fl, max}=600 mm, 650 nm) than the absorbance of cell culture media, while hydroxy-derivatives of compounds such as perylene diimide 2 or perylene monoimide 3 may absorb and emit at even longer wavelengths in some instances. For example, a hydroxy-substituted perylene monoimide is estimated to shift longer wavelengths by about 20 nm relative to an unsubstituted perylene monoimide.

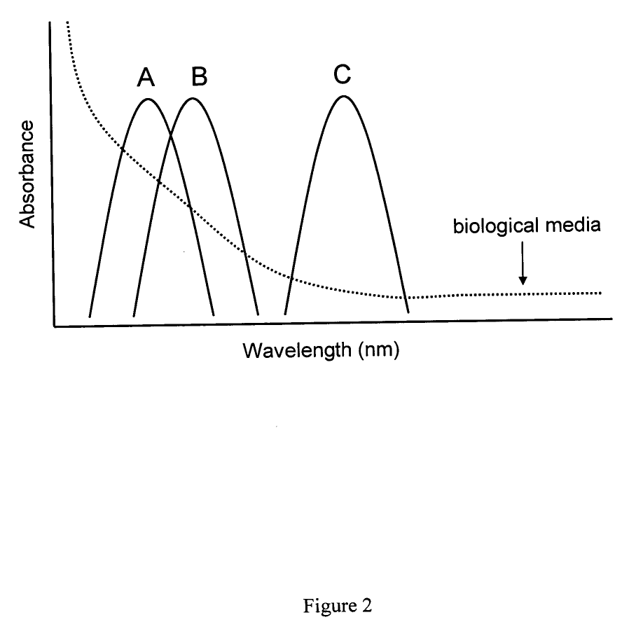

FIG. 2 shows the projected relative absorption spectra for compounds of the invention that include at least five fused organic rings with significant conjugation, the compounds able to be protonated or deprotonated, for example, pH indicator molecules, having increasingly larger aromatic cores, but is not representative of spectra measured with specific molecules or samples. For example, spectrum A represents the absorbance of an indicator molecule comprising four fused rings, such as a pyrene core, which is substantially masked by the absorbance of the background biological media (dotted line). Spectrum B, which represents the absorbance for an indicator molecule comprising five fused rings, such as a perylene core, exhibits an absorbance shifted to longer wavelengths resulting in a more clearly observable signal. A more dramatic shift to longer wavelengths can be observed by spectrum C, which represents the absorbance for an indicator molecule comprising five fused rings further substituted with electron-withdrawing functional groups, such as a perylene monoimide core. With increasing size of the aromatic core, the absorption spectrum is shifted to longer wavelengths, effectively separating the absorbance of the pH indicator molecule from the absorbance of biological media and improving the effectiveness of the indicator. All of the spectra of FIG. 2 are prophetic, i.e., they are representative of the expected behavior of molecules serving as indicators in accordance with the invention.

In addition to the spectral shifts, in certain embodiments, the size of the aromatic core of the molecule may affect the Kₐ of the molecule, which may alter or increase the pH range over which the indicator is useful. Generally, more extended aromatic systems may lower the Kₐ.

In one set of embodiments, the chemical compound of the invention can be readily covalently attached to another molecule such as a polymer via a suitable functional group, e.g. a polymerizable or crosslinkable group such as acrylate, methacrylate, methyl methacrylate, and the like. In this way, the molecule can be conveniently immobilized with respect to another molecule, polymer, or article. For example, the molecule can be covalently attached to a sensor so that the molecule can serve the purpose of indicating pH of a species to which the sensor (at least the region of the sensor at which the molecule is immobilized) is exposed. Such functional groups for immobilization are well known to those or ordinary skill in the art. In another embodiment, the chemical compound can be attached to another entity non-covalently, e.g. via hydrogen bonding, van der Waals interactions, etc. Examples of attachment including covalent linkage via reaction of sulfonyl groups and/or related groups, thiol-containing functional groups' adherence to a gold surface of an article, etc.

In another set of embodiments, attached functional groups may shift the Kₐ of the chemical compound in predictable ways, making the molecule more sensitive (i.e. have more response per pH unit) in a given pH range. Generally, electron-withdrawing functional groups will increase the Kₐ, while electron-donating groups will decrease the Kₐ.

In another aspect, the invention provides a method of determining pH. One embodiment of this aspect involves providing a chemical compound comprising at least five fused rings as described herein exposing the compound to an environment at a particular pH, and determining an interaction of the chemical compound with electromagnetic radiation indicative of the particular pH. The chemical compound, in this aspect, can be selected from any of the compound described herein, or other compounds falling within this definition.

In the method, the process of exposing the chemical compound to an environment at a particular pH encompasses a variety of individual techniques, including suspending or dissolving the molecule in a fluid (typically an aqueous fluid) where the pH of the fluid is desirably determined, immobilizing the compound on a surface and exposing the surface to a sample (e.g., a fluid sample), the pH of which is desirably determined, or the like. Those of ordinary skill in the art will recognize a wide variety of techniques that fall within this particular process.

This embodiment also involves determining an interaction of the chemical compound with electromagnetic radiation. Again, those of ordinary skill in the art will understand the wide variety of determinations encompassed by this technique. At the outset, the electromagnetic radiation can be of any specific radiation or any range of radiation, such as visible light, ultraviolet light, infrared radiation, etc. Typically, for pH indicators, this electromagnetic radiation is in the visible range, so that determination can be made easily by a human without resort to instrumentation. The invention is useful in this regime, but is not limited to this regime. The “interaction” of the compound with electromagnetic radiation is defined to include absorption and/or emission of the radiation and/or any other interaction in a way that changes in a determinable manner based upon pH (the position with respect to the protonation/deprotonation equilibrium of Eq. 1). For example, pH may affect the absorption wavelength of the compound, the emission wavelength of the compound, or both. Alternatively, or in addition, pH can affect the intensity or amount of absorption or emission of the electromagnetic radiation by the compound.

Techniques for measuring interaction of electromagnetic radiation with molecules are well known to those or ordinary skill in the art and can involve single measurement, ratiometric measurement or the like. In a single measurement, typically, the intensity of an emission or absorption wavelength band is measured alone. In a ratiometric measurement, an increase in intensity of one wavelength band is measured simultaneously with the corresponding decrease in intensity of another wavelength band. Ratiometric determination can be more sensitive. Both techniques and other known techniques, are included within methods of the invention.

In one set of embodiments, absorption and/or emission of electromagnetic radiation by a composition of the invention takes place, to a significant extent, within the visible region, specifically, at a peak wavelength of at least 450 nanometers for absorption and at least 550 nanometers for emission, leading to less background fluorescence from biological fluids and cells. In other embodiments both absorption and emission takes place at greater than 450 nanometers or greater than 550 nanometers.

In another set of embodiments, a change in the protonation/deprotonation equilibrium may cause a measurable change in an optical property of the compound. Non-limiting examples of such optical properties include one or more of: changes in the molar absorptivity at a specific wavelength of light of the compound, changes in the wavelength of light of maximum absorptivity of the compound, changes in the quantum yield of emission of the compound, changes in the wavelength of light of maximum emission intensity of the compound, and/or changes in the emission lifetime of the compound.

Those of ordinary skill in the art are well aware of the phenomena discussed above and how to determine them. If not easily determinable by the naked human eye, they can be determined by instrumentation such as absorption spectroscopy, fluorescence spectroscopy, etc.

As noted above, methods of the invention can be carried out in a variety of settings. One example of such a setting is a microfluidic environment in which a chemical, biochemical, or biological process is carried out and where at least one aspect of the process is desirably monitored with respect to pH. Specific examples include techniques for cell culturing where pH may be adjusted, or at least determined for a variety of reasons. For example, cell culture techniques may involve determination of particular conditions (e.g., pH and optionally others) under which a cell produces particular products. Another exemplary process involves the screening of drugs against cells and/or their products to identify effective compositions for potential therapeutic use. In these and other microfluidic techniques, it can be desirable to measure pH at one or more locations in the microfluidic device, optionally in conjunction with a controller to control pH. Techniques such as those described in International Patent Application No. PCT/US03/25907, filed Aug. 19, 2003, entitled “Systems and Methods for Control of pH and Other Reactor Environmental Conditions,” by Miller, et al., published as WO 2004/016729 on Feb. 26, 2004, and incorporated herein by reference can be used in conjunction with the compositions and methods of the present invention.

In one set of embodiments, compositions of the invention are relatively easily derivatizable (modifiable by chemical reaction) to form new compounds with different absorption and/or emission wavelengths. In this set of embodiments compositions of the invention, such as those illustrated above as molecules 1-26, can be readily altered by replacing an “R” group, i.e., substituting a group pendent from the fused ring system (or pendant from such a group) such that an atom directly bonded to a carbon atom of the fused ring system (or bonded to a group so bonded, etc.) is replaced. This can result in adjustments to the molecule to optimize its sensitivity to pH determination in a variety of media, with a variety of background absorption/emission characteristics that otherwise might be interfering. As used herein, “readily derivatized,” “easily derivatizable,” and like terminology means derivatized through standard organic chemical processes involving generally less than two reactive steps, or less than three or four reactive steps in other embodiments. These processes can involve, for example, halogenation (e.g., chlorination, bromination, or the like) of rings or side chains of these systems, for example with FeCl₃, elemental halogens with heat and/or light, standard bromination reactions, free radical reactions involving heat, or light, or the like. Those of ordinary skill in the art will understand the meaning of “readily derivatizable” in this context.

Readily derivatizable compounds of the invention can also be derivatized to adjust solubility in a way that can or may not necessarily, affect the pH sensitivity of the composition. For example, a molecule can be made more or less hydrophilic so as to increase or decrease solubility in a aqueous environment, in a way that may or may not effect the wavelength sensitivity of the composition (absorption and/or emission wavelengths) and/or absorption/emission sensitivity (intensity).

In one set of embodiments, compounds of the invention are readily derivatizable to include at least one functional group which increases the permeability of the compound to a cell membrane, relative to a reference compound similar to the compound except without the at least one functional group.

In one set of embodiments, compounds of the invention are readily derivatizable to alter their pH indicating wavelength or range by being readily derivatizable in a way that involves breaking and re-forming at least one covalent bond that is separated from a carbon or other atom, which defines the fused ring system of the molecule, by no more than 5 atoms, or no more than 4, 3, 2, or 1 atom. In one set of embodiments, these compounds are readily derivatizable to alter their pH indicating wavelength by being readily derivatizable in a way that involves breaking and re-forming at least one covalent bond directly attached to the fused ring system of the molecule. The distance from the fused ring system at which derivatization occurs, in combination with a change in electron donating or withdrawing characteristic of the derivatized group, will affect the shift in pH indicating wavelength or range, and selection of appropriate groups and distances from the ring system are controllable by those of ordinary skill in the art to achieve a desired result.

Compounds of the invention also can be readily derivatizable to alter their solubility and/or other properties, as discussed elsewhere herein, by being readily derivatizable at distances from the fused ring system as discussed immediately above. Derivatization to affect solubility and certain other characteristics can change independently of the pH-indicating electronic structure of the molecule without detriment to the invention. Accordingly, derivatization to affect solubility or another characteristic that can be independent of pH indicating wavelength or range can take place, in another set of embodiments, at any location relative to the fused ring system, so long as the desired effect is achieved.

The following documents describe generally, synthesis and uses of perylenes and derivatives, and knowledge from these references and other references available to those of ordinary skill in the art can be used in making, derivatizing, and using compounds of the present invention. All of these documents are incorporated by reference: Feiler, L.; Langhals, H.; Polborn, K. Leibigs Ann. 1995, 1229-1244; Quante, H.; Mullen, K. Angew. Chem. Int. Ed. Engl. 1995, 34, 1323-1325; Ahrens, et. al. Chem. Mater. 2003, 15, 2684-2686; Zhao, et. al. Tetrahedron Lett. 1999, 40, 7047-7050; Miller, et. al. Chem. Phys. 2002, 275, 167-183. Some of these reactions include adding cyano groups to perylene to lower oxidation potential, adding pyrrole groups to lower absorbance maximum of a molecule (reduce the HOMO-LUMO gap), changing absorbance, fluorescence maxima and oxidation potential of various molecules via pyrrole vs. piperidine-substituted perylenes, etc.

### EXAMPLE 1 (PROPHETIC)

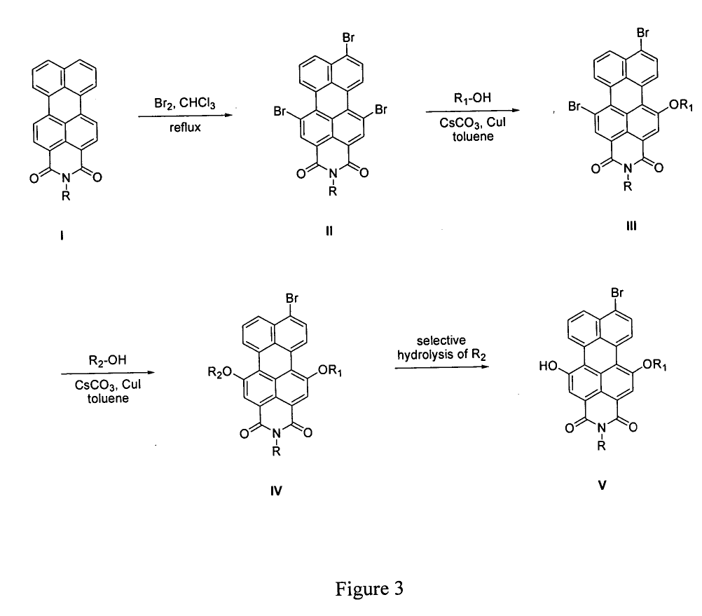

The synthetic scheme illustrated in FIG. 3 details an example of a method by which molecules of the present invention may be synthesized. Compound IV is prepared using techniques as described previously (see Miller, et. al., Chem. Phys. 2002, 275, 167-183) wherein the perylene monoimide I is brominated in three positions to form compound II. Conversion of one bromide to a functional group capable of covalent linkage to a polymer is achieved by treatment with R₁—OH and cesium carbonate in the presence of CuI, affording compound III. Examples of R₁ may be acrylate, methacrylate, methyl methacrylate, and the like. Conversion of another bromide to a functional group which is capable of protonation and deprotonation based upon the pH of a surrounding medium, such as a hydroxyl group, is performed in two steps. First, treatment with R₂—OH and cesium carbonate in the presence of CuI gives compound IV, wherein R₂ may be a function group capable of undergoing hydrolysis. Second, selective hydrolysis of R₂ by methods known in the art affords the final product V. While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

All definitions, as defined and used herein, should be understood to control over dictionary definitions, definitions in documents incorporated by reference, and/or ordinary meanings of the defined terms.

It should also be understood that, unless clearly indicated to the contrary, in any methods claimed herein that include more than one act, the order of the acts of the method is not necessarily limited to the order in which the acts of the method are recited.

The indefinite articles “a” and “an,” as used herein in the specification and in the claims, unless clearly indicated to the contrary, should be understood to mean “at least one.”

As used herein, “or” should be understood to mean inclusively or, i.e., the inclusion of at least one, but including more than one, of a number or list of elements. Only terms clearly indicated to the contrary, such as “only one of” or “exactly one of,” will refer to the inclusion of exactly one element of a number or list of elements.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims (as well as in the specification above), all transitional phrases such as “comprising”, “including”, “carrying”, “having”, “containing”, “involving”, “composed of”, “made of”, “formed of” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

1. A composition comprising a compound including at least five fused molecular rings, the compound able to be protonated or deprotonated as a result of change in pH of a medium to which the compound is exposed.

2. The composition of claim 1, wherein protonation or deprotonation causes a measurable change in interaction of electromagnetic radiation with the compound.

3. The composition of claim 1, wherein protonation or deprotonation of the compound causes a measurable change in an optical property of the compound.

4. The composition of claim 3, wherein the optical property is detectable to the unaided human eye.

5. The composition of claim 3, wherein the optical property includes molar absorptivity at a specific wavelength of light.

6. The compound of claim 3, wherein the optical property includes wavelength of light of maximum absorptivity.

7. The compound of claim 3, wherein the optical property includes quantum yield of emission.

8. The compound of claim 3, wherein the optical property includes wavelength of light of maximum emission intensity.

9. The compound of claim 3, wherein the optical property includes emission lifetime.

10. The compound of claim 1, wherein the compound includes at least one functional group.

11. The compound of claim 10, wherein the at least one function group is electron-donating.

12. The compound of claim 10, wherein the at least one function group is electron-withdrawing.

13. The compound of claim 10, wherein the at least one functional group modifies an optical property of the compound, relative to a reference compound similar to the compound except without the at least one functional group.

14. The compound of claim 10, wherein the at least one functional group modifies an optical property of the compound, relative to a reference compound free of functional groups.

15. The compound of claim 13, wherein the optical property includes molar absorptivity at a specific wavelength of light.

16. The compound of claim 13, wherein the optical property includes wavelength of light of maximum absorptivity.

17. The compound of claim 13, wherein the optical property includes quantum yield of emission.

18. The compound of claim 13, wherein the optical property includes wavelength of light of maximum emission intensity.

19. The compound of claim 13, wherein the optical property includes emission lifetime.

20. The compound of claim 10, wherein the at least one functional group causes a shift in pKa relative to a reference compound similar to the compound except without the at least one functional group.

21. The compound of claim 10, wherein the at least one functional group increases the solubility of the compound in water relative to a reference compound similar to the compound except without the at least one functional group.

22. The compound of claim 10, wherein the at least one functional group is a polymerizable or crosslinkable group.

23. The compound of claim 10, wherein the at least one functional group is able to covalently bond the compound to a substrate.

24. The compound of claim 23, wherein the substrate is a polymer.

25. The compound of claim 23, wherein the substrate is an article having a surface.

26. The compound of claim 23, wherein the substrate is another chemical compound.

27. The compound of claim 10, wherein the at least one functional group increases the permeability of the compound to a cell membrane, relative to a reference compound similar to the compound except without the at least one functional group.

28. The compound of claim 10, wherein the at least one function group is at least one of an acrylamide, a carboxylic acid, and activated ester of a carboxylic acid, a hydroxyl, an aldehyde, an alkyl halide, a sulfonate, an amine, an anhydride, an aniline, an aryl halide, an azide, an aziridine, a boronate, a carbodiimide, and epoxide, a glycol, an haloacetamide, a halotrazine, a hydrazine, a hydroxylamine, an isothiocyanate, an isocyanate, a thiocarbamate, a ketone, a maleimide, a sulfonyl halide, or a thiol group.

29. The compound of claim 10, wherein the at least one function group is L-Rₓ, L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
Rₓ being a succinimidyl ester.

30. The compound of claim 10, wherein the at least one functional group is sulfomethyl, halomethyl, C₁-C₁₈ or C₁-C₁₈ perfluoroalkyl.

31. The compound of claim 10, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being an amino acid, a tyramine, a peptide, a protein, a monosaccharide, a polysaccharide, an ion-complexing moiety, a nucleoside, a nucleotide, an oligonucleotide, a nucleic acid, a hapten, a drug, a lipid, a phospholipid, a lipoprotein, a lipopolysaccharide, a liposome, a lipophilic polymer, a polymeric microparticle, an animal cell, a plant cell, a bacterium, a yeast, or a virus.

32. The compound of claim 10, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being an amino acid, a peptide, a protein, a nucleotide, an oligonucleotide, a nucleic acid, a monosaccharide, a polysaccharide, or a drug.

33. The compound of claim 10, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being a peptide or a protein.

34. The compound of claim 10, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being a metallic or semiconductor nanoparticle.

35. The compound of claim 10, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being a fullerene or carbon nanotube.

36. The compound of claim 10, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} comprises one or more additional dye compounds, which may be the same or different.

37. A method comprising:
providing a chemical compound comprising at least five fused molecular rings;
exposing the molecule to an environment at a pH: and
determining the pH of the environment by determining an interaction of the chemical compound with electromagnetic radiation.

38. The compound of claim 1, wherein the compound has a structure:

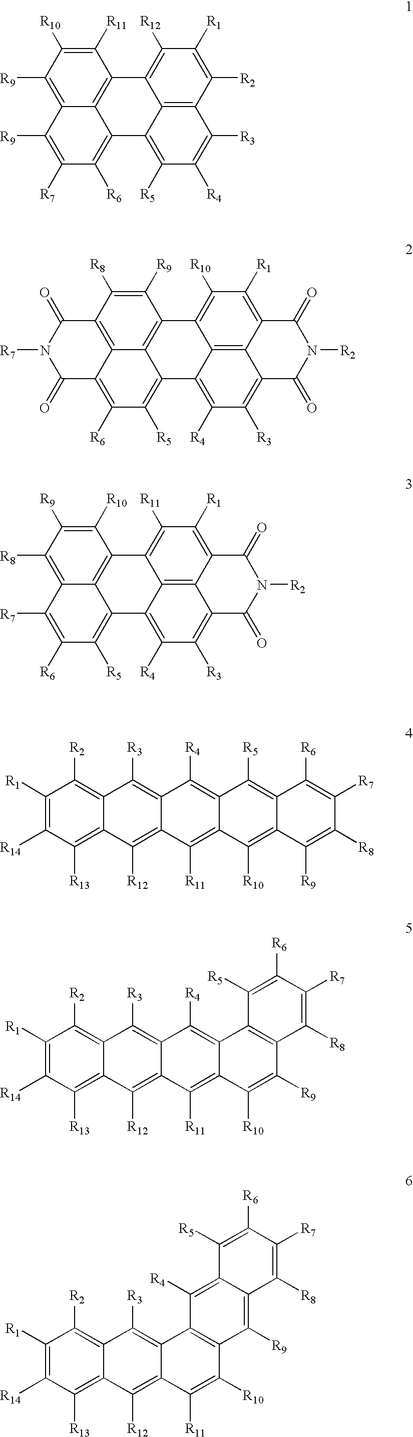

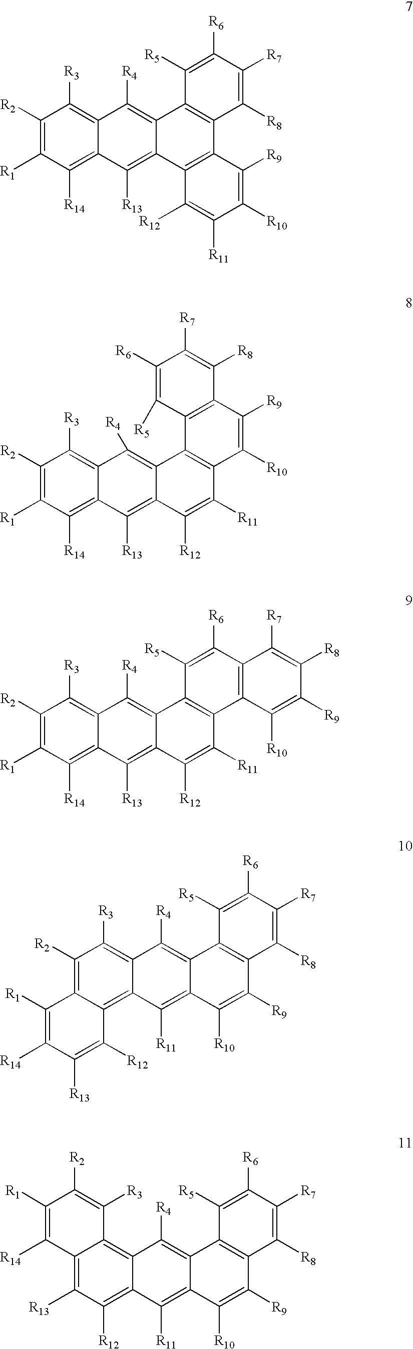

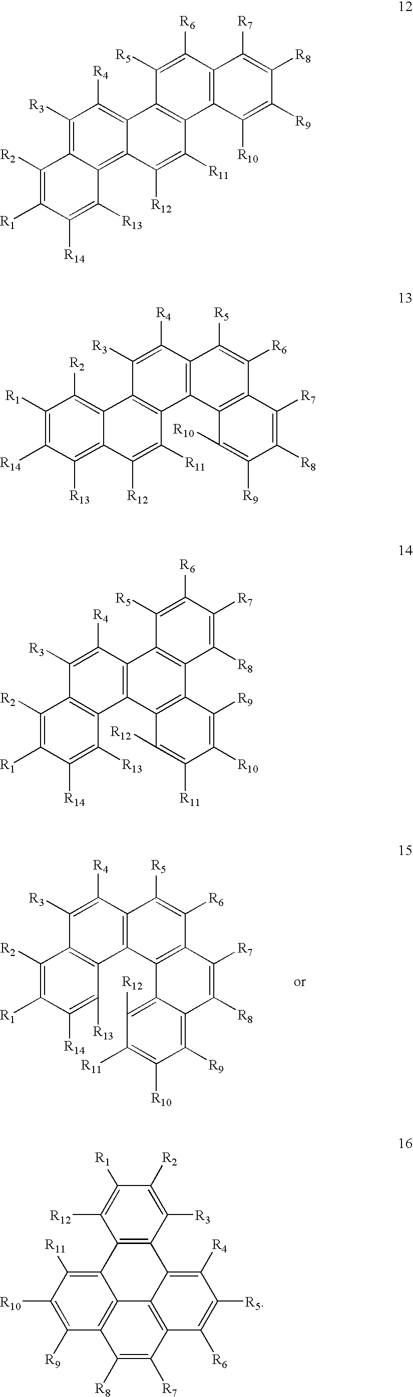

39. The compound of claim 38, wherein at least one R is at least one of an acrylamide, a carboxylic acid, and activated ester of a carboxylic acid, a hydroxyl, an aldehyde, an alkyl halide, a sulfonate, an amine, an anhydride, an aniline, an aryl halide, an azide, an aziridine, a boronate, a carbodiimide, and epoxide, a glycol, an haloacetamide, a halotrazine, a hydrazine, a hydroxylamine, an isothiocyanate, an isocyanate, a thiocarbamate, a ketone, a maleimide, a sulfonyl halide, or a thiol group.

40. The compound of claim 38, wherein at least one R is -L-Rₓ,
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds, and
Rₓ being a succinimidyl ester.

41. The compound of claim 38, wherein at least one R is sulfomethyl, halomethyl, C₁-C₁₈ or C₁-C₁₈ perfluoroalkyl.

42. The compound of claim 38, wherein at least one R is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being an amino acid, a tyramine, a peptide, a protein, a monosaccharide, a polysaccharide, an ion-complexing moiety, a nucleoside, a nucleotide, an oligonucleotide, a nucleic acid, a hapten, a drug, a lipid, a phospholipid, a lipoprotein, a lipopolysaccharide, a liposome, a lipophilic polymer, a polymeric microparticle, an animal cell, a plant cell, a bacterium, a yeast, or a virus.

43. The compound of claim 38, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being an amino acid, a peptide, a protein, a nucleotide, an oligonucleotide, a nucleic acid, a monosaccharide, a polysaccharide, or a drug.

44. The compound of claim 38, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being a peptide or a protein.

45. The compound of claim 38, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being a metallic or semiconductor nanoparticle.

46. The compound of claim 38, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} being a fullerene or carbon nanotube.

47. The compound of claim 38, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds; and
S_{c} comprises one or more additional dye compounds, which may be the same or different.

48. The compound of claim 38, wherein the at least one functional group is L-S_{c},
L being a covalent linkage having 1-24 nonhydrogen atoms comprising one or more of C, N, O and S, and is comprised of any combination of single, double, triple or aromatic carbon-carbon bonds, carbon-nitrogen bonds, nitrogen-nitrogen bonds, carbon-oxygen bonds, and carbon-sulfur bonds; and
S_{c} comprises one or more additional compounds which may quench the fluorescence of the compound.

49. The compound of claim 38, wherein the at least one functional group is L-S_{c},
L being a covalent linkage having 1-24 nonhydrogen atoms selected from the group consisting of C, N, O and S and is composed of any combination of single, double, triple or aromatic carbon-carbon bonds, carbon-nitrogen bonds, nitrogen-nitrogen bonds, carbon-oxygen bonds, and carbon-sulfur bonds and
S_{c} comprises one or more additional compounds which may undergo energy transfer to or from compound.

50. A composition as in claim 1, the compound comprising at least five fused organic molecular rings having significant delocalization of pi-electron structure such that the compound absorbs and/or emit electromagnetic radiation significantly at wavelengths greater than 400 nm or 450 nanometers.

51. A composition as in claim 50, wherein the compound absorbs at greater than 400 nm or 450 nanometers with a molar absorptivity at at least 5000/mole.cm, and/or emits at greater than 400 nm or 450 nanometers with a quantum yield of at least 5%.

52. A composition as in claim 51, wherein the compound is a hydrocarbon-based compound optionally including heteroatoms.
